(19) United States
(12) Patent Application Publication
(10) Pub. No.: US 2021/0040445 A1
(43) Pub. Date: Feb. 11, 2021

(54) METHOD FOR PRODUCING TAU-RELATED DISEASE MODEL

(71) Applicant: Keio University, Tokyo (JP)

(72) Inventors: Mari NAKAMURA, Tokyo (JP); Seiji SHIOZAWA, Tokyo (JP); Hideyuki OKANO, Tokyo (JP)

(73) Assignee: Keio University, Tokyo (JP)

(21) Appl. No.: 16/984,241

(22) Filed: Aug. 4, 2020

(30) Foreign Application Priority Data
Aug. 6, 2019 (JP) 2019-144808

(51) Int. Cl. C12N 5/0793; C12N 5/079; C12N 5/00; G01N 33/50

(52) U.S. Cl. CPC C12N 5/0619; C12N 5/0618; C12N 5/0062; C12N 2503/02; C12N 2513/00; C12N 2506/45; C12N 2506/08; G01N 33/5058

(57) ABSTRACT

A method for producing a tau-related disease model is provided. The method includes three-dimensionally culturing pluripotent stem cells having a mutation in a Microtubule Associated. Protein Tau (MAPT) gene to form a nerve organoid, and dissociating the nerve organoid into single cells and performing two-dimensional adherent culture to obtain neurons, in which the neurons are a tau-related disease model.

### TECHNICAL FIELD

The present invention relates to a method for producing a tan-related disease model. More specifically, the present invention relates to a method for producing a tau-related disease model, a tau-related disease model, and a method for screening a preventive or therapeutic agent for a tau-related disease. Priority is claimed on Japanese Patent Application No. 2019-144808, filed on Aug. 6, 2019, the content of which is incorporated herein by reference.

### BACKGROUND ART

Frontotemporal dementia (FTD) is one of the tau-related diseases, and is a neurodegenerative disease caused by a mutation in the Microtubule Associated Protein Tau (MAPT) gene, which encodes tau protein.

Over 50 mutations in the MAPT gene have been reported to induce frontotemporal dementia, and the disease phenotype is known to differ among patients with different mutations. At present, no effective treatment is known for patients with frontotemporal dementia, and the development of a tau-related disease model has been required for the development of treatment techniques.

For example, Non-Patent Literature 1 reports that pathological aspects of frontotemporal dementia patients could be clarified as a result of analysis using transgenic mouse models and the brain of postmortem patients.

### CITATION LIST

### Non-Patent Literature

Non-Patent Literature 1

Denk F. and Wade-Martins R., Knock-out and transgenic mouse models of tauopathies., Neurobiol Aging, 30 (1), 1-13, 2009.

### SUMMARY OF INVENTION

### Technical Problem

However, the transgenic mouse models disclosed in Non-Patent Literature 1 cannot adequately reflect the pathological aspects of human tau-related diseases due to species differences and overexpression of tau transgenes. Further, the brains of postmortem patients cannot be used as a model for the onset and progression of tau-related diseases. Accordingly, the present invention aims to provide a technique for producing a tau-related disease model.

### Solution to Problem

The present invention includes the following aspects:

[1] A method for producing a tau-related disease model, including the steps of: three-dimensionally culturing pluripotent stem cells having a mutation in the MAPT gene to form a nerve organoid; and dissociating the nerve organoid into single cells and performing two-dimensional adherent culture to obtain neurons, in which the neurons are a tau-related disease model.

[2] The method for producing according [1], in which the mutation is one or a plurality of mutations present in exons 9 to 13 or intron 10.

[3] The method for producing according to [2], in which the mutation preset exons 9 to 13 is a mutation that encodes a mutant tau protein selected from the group consisting of K257T, I260V, G272V, N279K, K280Δ, L284L, S285R, N296H, P301L, P301S, S305N, S303S, S305S, V337M, E342V, G389R and R406W, and the mutation present in intron 10 is a mutation in any one or a plurality of bases at nucleotide positions 1 to 20 from the 5′-end of intron 10.

[4] A tau-related disease model including neurons having a mutation in the MAPT gene, in which the neurons are significantly different in the degree of phosphorylation of tau protein, degree of fragmentation of tau protein, degree of axonal degeneration or axonal transport function, relative to wild-type neurons.

[5] The tau-related disease model according to [4], which is produced by the method for producing according to any one of [1] to [3].

[6] A method for screening a preventive or therapeutic agent for a tau-related disease, the method including the steps of: culturing the tau-related disease model according to [4] or [5] in the presence of a test substance; and measuring the degree of phosphorylation of tau protein, degree of fragmentation of tau protein, degree of axonal degeneration or axonal transport function of the tau-related disease model, in which a significant difference in the measured degree of phosphorylation of tau protein, degree of fragmentation of tau protein, degree of axonal degeneration or axonal transport function, relative to that in the absence of the test substance, is an indication that the test substance is a preventive or therapeutic agent for a tau-related disease.

### Advantageous Effects of Invention

According to the present invention, a technique for producing a tau-related disease model can be provided.

### BRIEF DESCRIPTION OF DRAWINGS

FIGS. 1A and 1B show schematic views of structures of targeting vectors used in Experimental Example 2.

FIGS. 2A to 2C show traces of the results of nucleotide sequence analysis of the tau gene of iPS cells prepared in Experimental Example 2.

FIG. 3 shows a schematic view of a schedule of differentiation from iPS cells to neurons in Experimental Example 3.

FIG. 4 shows fluorescence micrographs of the representative results of immunostaining of tau-related disease models prepared in Experimental Example 3.

FIGS. 5A to 5E show graphs of the results of measuring the proportion of each type of marker-positive cells in the tau-related disease models prepared in Experimental Example 3. FIG. 5A shows the results for MAP2, FIG. 5B shows the results for βIII-tubulin, FIG. 5C shows the results for TBR1, FIG. 5D shows the results for NEUN, and FIG. 5E shows the results for FOXG1.

FIG. 6A shows photographs of the results of examining the phosphorylation of tau protein in Experimental Example 4. FIGS. 6B and 6C are graphs quantifying the results of FIG. 6A. FIG. 6D shows a photograph of the results of examining the phosphorylation of tau protein in Experimental Example 4.

FIG. 7A shows photographs of the results of Western blotting sing a Tau12 antibody in Experimental Example 5. FIG. 7B is a graph quantifying the amounts of fragmented tau protein based on the results of FIG. 7A.

FIG. 8A shows fluorescence micrographs of the representative results of immunostaining in Experimental Example 6. FIG. 8B is a graph quantifying proportions of tau protein localized in dendrites, and is a graph quantifying proportions of tau protein localized in axons based on the results of the immunostaining in Experimental Example 6.

FIG. 9 shows fluorescence micrographs of the representative results of immunostaining in Experimental Example 6.

FIG. 10A shows fluorescence micrographs of the representative results of immunostaining in Experimental Example 7. FIG. 10B shows a graph of the results of measuring the number of neurite puncta based on the results of immunostaining

Experimental Example 7.

FIG. 11A shows representative fluorescence micrographs in Experimental Example 8. FIG. 11B shows a graph of the results of measuring the number of mitochondria present on axons based on the fluorescence micrographs in Experimental Example 8.

FIG. 12 shows a graph of the results of measuring the proportion of anterograde, retrograde, and stationary mitochondria in Experimental Example 8.

### DESCRIPTION OF EMBODIMENTS

[Method for Producing Tau-Related Disease Model]

In one embodiment, the present invention provides a method for producing a tau-related disease model, in which the method includes the steps of; three-dimensionally culturing pluripotent stem cells having a mutation in the MAPT gene to form a nerve organoid and dissociating the nerve organoid into single cells and performing two-dimensional adherent culture to obtain neurons, in which the neurons can be used as a tau-related disease model. In the present specification, examples of the tau-related disease include frontotemporal dementia and Alzheimer's disease.

As will be described later in Examples, the tau-related disease model produced by the method for producing of the present embodiment can be used for elucidation of the mechanism of onset and progression of a tau-related disease, and for screening a preventive or therapeutic agent for a tau-related disease.

Examples of pluripotent stein cells include embryonic stem cells (ES cells) and induced pluripotent stem cells (iPS cells). The pluripotent stem cells are preferably human cells.

The pluripotent stem calls having a mutation in the MAPT gene may be pluripotent stem cells derived from a patient with a tau-related disease or pluripotent stem cells derived from a healthy person in which a mutation is artificially introduced into the MAPT gene. Alternatively, the pluripotent stem cells may be pluripotent cells obtained by further artificially introducing the MAPT gene into pluripotent stem cells derived from a patient with a tau-related disease.

The NCBI accession number of the nucleotide sequence of the human MAPT genomic DNA is NC_000017.11. In the method for producing of the present embodiment, the mutation of the MAPT gene is not particularly limited as long as the mutation is associated with a tau-related disease, and is preferably, for example, one or a plurality of mutations present in exons 9 to 13 or intron 10.

More specific examples of the mutations present in exons 9 to 13 include a mutation that encodes a mutant tau protein such as K257T, I260V, G272V, N279K, K280Δ, L284L, S285R, N296H, P301L, P301S, S305N, S303S, S305S, V337M, E342V, G389R and R406W. Among these mutations, L284L, S303S, and S305S are mutations that are not involved in amino acid substitution and cause abnormal splicing resulting from codon changes.

Further, examples of the mutations present in intron 10 include a mutation in any one or a plurality of bases at nucleotide positions 1 to 20 from the 5′-end of intron 10. More specific examples of the mutations present in intron 10 include Ex10+3, Ex10+12, Ex10+13, Ex10+14, Ex10+16, Ex10+19, and Ex10+29.

The three-dimensional culture of pluripotent stein cells may be carried out by forming aggregates (embryoid bodies) of pluripotent stem cells on a U-bottom or V-bottom plate having low cell adhesiveness and culturing the aggregates of pluripotent stem cells or may be carried out by embedding pluripotent stem cells in an extracellular matrix and culturing the embedded pluripotent stem cells.

The extracellular matrix (Extracellular Matrix, ECM) to be used may be a commercially available matrix such as Matrigel (registered trademark, Corning Inc.), Cellmatrix (Nitta Gelatin), extracellular matrix protein (Thermo Fisher Scientific Inc.) or ProNectin (Sigma Inc.), for example.

Alternatively, an ECM may be prepared and used. Examples of the ECM preparation method include a method of culturing ECM-producing cells in a culture vessel, removing the cells, and using the ECM coated on a surface of the culture vessel. Examples of ECM-producing cells include chondrocytes that mainly produce collagen and proteoglycans, fibroblast cells that mainly produce collagen type IV collagen, laminin, interstitial procollagens and fibronectin; and colonic myofibroblasts that mainly produce collagens (type I, III and V), chondroitin sulfate proteoglycan, hyaluronic acid, fibronectin and tenascin-C.

An organoid is a structure formed by accumulating cells and has a structure and function similar to an organ in a living body. In recent years, research for preparing various types of organoids from pluripotent stem cells has been actively conducted, and, for example, nerve organoids (brain organoids), intestinal organoids, liver organoids, kidney organoids and the like have been prepared.

In regards to methods for producing the present embodiment, the method of forming a nerve organoid from pluripotent stem cells is not particularly limited, and examples thereof include three-dimensionally culturing pluripotent stem cells in the presence of a TGF-βinhibitor and a Wnt inhibitor.

Examples of TGF-β inhibitors that can be used include SB-431542 (CAS number: 301836-41-9), A83-01 (CAS number: 909910-43-6) and RepSox (CAS number: 446859-33-2).

Examples of Wnt inhibitors that can be used include IWP-2 (CAS number: 686770-61-6), IWP-3 (CAS number: 687561-60-0), IWP-4 (CAS number: 686772-17-8) and XAV-939 (CAS number: 284028-89-3).

The three-dimensional culture of pluripotent stein cells is preferably performed for, for example, 20 days or more, for example, 25 days or more, for example, 30 days or more. There is no particular upper limit on the period of three-dimensional culture. As a result of the three-dimensional culture, a nerve organoid can be obtained.

Subsequently, the nerve organoid is dissociated into single cells. The method of dissociating the nerve organoid is not particularly limited, and examples thereof include physical methods and enzymatic treatment methods, but the enzymatic treatment method is preferable from the viewpoint of not damaging cells. Examples of the enzyme that can be used in the enzymatic treatment method include TrypLE Express (Thermo Fisher Scientific Inc.), TrypLE Select (Thermo Fisher Scientific Inc.), papain, trypsin, collagenase and dispase I.

The dissociation of the nerve organoid is preferably performed until the nerve organoid is dissociated into single cells, but it does not have to be completely dissociated into single cells. Specifically, the nerve organoid may be dissociated into, for example, 2 to 10, for example, 2 to 8, for example, 2 to 5 cell aggregates.

Subsequently, the cells obtained by dissociating the nerve organoid are two-dimensionally adherently cultured. The two-dimensional culture is a common culture using a plate, a dish or the like. As will be described later in Examples, a highly pure population of neurons can be obtained by performing two-dimensional culture.

The two-dimensional culture is preferably performed for, for example, 20 days or more, for example, 25 days or more, for example, 30 days or more. There is no particular upper limit on the period of two-dimensional culture. The two-dimensional culture may be carried out in a medium used for usual neuron culture. Examples of such a medium include Neurobasal medium (Thermo Fisher Scientific Inc.) and BrainPhys medium (Stem Cell Technologies).

As will be described later in Examples, the neurons after two-dimensional culture sufficiently reflect the pathological aspects of tau-related diseases and can thus be used as a tau-related disease model.

[Tau-Related Disease Model]

In one embodiment, the present invention provides a tau-related disease model including: neurons having a mutation in the MAPT gene, in which the neurons significantly different in the degree of phosphorylation of tau protein, degree of fragmentation of tau protein, degree of axonal degeneration or axonal transport function, relative to wild-type neurons. The tau-related disease model of the present embodiment can be produced, for example, by the method for production described above.

The tau-related disease model of the present embodiment has a mutation in the MAPT gene. The mutation of the MAPT gene is the same as described above.

As will be described later in Examples, the tau-related disease model of the present embodiment is significantly different in the degree of phosphorylation of tan protein, degree of fragmentation of tau protein, degree of axonal degeneration and axonal transport function, relative to wild-type neurons, reflecting the pathological aspects of tau-related diseases.

The degree of phosphorylation of tau protein may be higher or lower than that of wild-type neurons, depending on the type of MAPT gene imitation. For example, when the tau-related disease model has an R406W mutant tau protein, the degree of phosphorylation of tau protein is significantly reduced.

The phosphorylation of tau protein can be measured by Western blotting or the like using an anti-phosphorylated tau antibody. Further, the fragmentation of tau protein can be measured by Western blotting or the like using an anti-tau antibody.

Further, the axonal degeneration can be measured based on, for example, an immunostained image of neurons. For example, as wilt be described later in Examples, when the tau-related disease model has an R406W mutant tau protein, a large number of small puncta are observed in the axons of neurons.

It is known that protein is scarcely synthesized in the axons of neurons. Therefore, most proteins required in axons and synaptic region to be synthesized in the cell body and then transported into axons. This substance transport in axons is called axonal transport. Axonal transport, in which motor proteins (kinesin and dynein) carry various organdies and protein complexes bidirectionally along microtubules, plays a fundamental and important role in neuronal survival, morphogenesis and functional expression. In axons, the microtubules are aligned, with the + (plus) end facing the axon terminal and the (minus) end facing the cell body. Anterograde transport is transport towards the + end of microtubules. On the other hand, retrograde transport is transport toward the end of the microtubules.

The axonal transport function can be evaluated by measuring the number of mitochondria contained in an axon per unit length by, for example, microscopic observation. Alternatively, the axonal transport function can be evaluated by measuring the proportion of anterograde, retrograde and stationary mitochondria or the like by live image analysis.

[Method for Screening for Preventive or Therapeutic Agent for Tau-Related Disease]

In one embodiment, the present invention provides a method for screening a preventive or therapeutic agent for a tau-related disease, in which the method includes the steps of: culturing the tau-related disease model described above in the presence of a test substance; and measuring the degree of phosphorylation of tau protein, degree of fragmentation of tau protein, degree of axonal degeneration or axonal transport function of the tan-related disease model, in which a significant difference in the measured degree of phosphorylation of tan protein, degree of fragmentation of tau protein, degree of axonal degeneration or axonal transport function, relative to that in the absence of the test substance, is an indication that the test substance is a preventive or therapeutic agent for a tau-related disease.

The test substance is not particularly limited, and examples thereof include a natural compound library, a synthetic compound library, an existing drug library and a metabolite library.

In the screening method of the present embodiment, the phosphorylation of tau protein, the fragmentation of tau protein, the axonal degeneration and the axonal transport function are the same as those described above. When the degree of phosphorylation of tau protein, degree of fragmentation of tau protein, degree of axonal degeneration and axonal transport function measured in the presence of the test substance are significantly changed relative to those in the absence of the test substance, it can be determined that the test substance can be a preventive or therapeutic agent for a tau-related disease.

### EXAMPLES

Next, the present invention will be described in more detail with reference to Examples, but the present invention is not limited to the following Examples.

### Experimental Example 1

(Preparation of MAPT R406W iPS cells)

MAPT R406W iPS cells were established frons two Japanese patients with pond dementia in the same pedigree (hereinafter, sometimes referred to as “patient #1” and “patient #2”). iPS cells were prepared using an episomal vector. The initial symptom of these patients was memory impairment. DNA sequencing confirmed that the imitations in the MAPT gene of these patients were heterozygous. There were no mutations other than R406W in the MAPT gene.

Further, MAPT R406W iPS cells were also established in the same manner as above from a patient in a pedigree different from patients #1 and #2 (hereinafter, sometimes referred to as “patient #3”). The mutation in the MAPT gene of patient #3 was also confirmed to be heterozygous.

### Experimental Example 2

(Preparation of Isogenic Line of iPS Cell Line by Genome Editing)

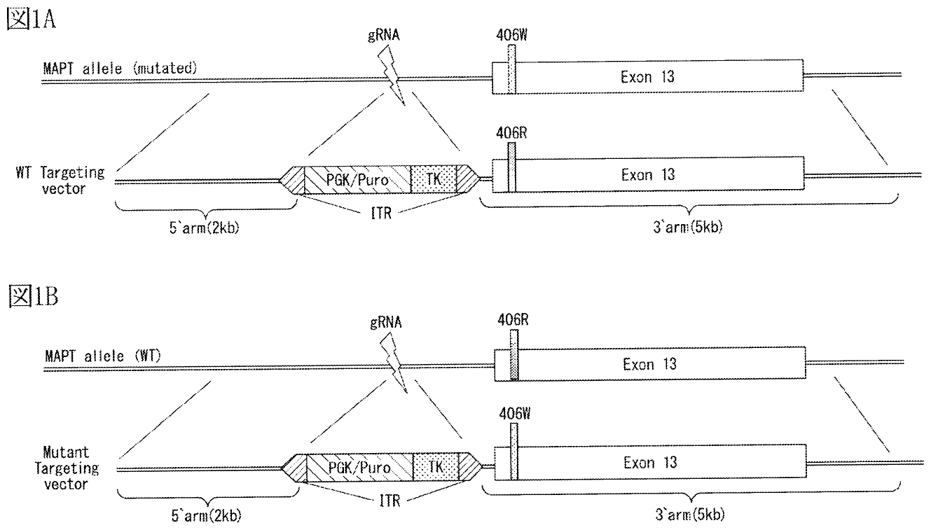

By genome editing using the CRISPR/Cas9 system, isogenic lines of wild-type iPS cells and homozygous mutant iPS cells were prepared from each type of the iPS cells prepared in Experimental Example 1. The targeting vector designed and used was a vector in which the 3′-arm contains a mutated site of the MAPT gene and a drug-selectable selection cassette is provided between the 3′-arm and the 5′-arm. The selection cassette was designed to be removable by placing PiggyBac ITRs at both terminals. FIGS. 1A and 1B show schematic views of structures of the targeting vectors. FIG. 1A is a schematic view of a targeting vector for recombining the mutant MAPT locus with a wild-type, and FIG. 1B is a schematic view of a targeting vector for recombining the wild-type MAPT locus with a mutant type.

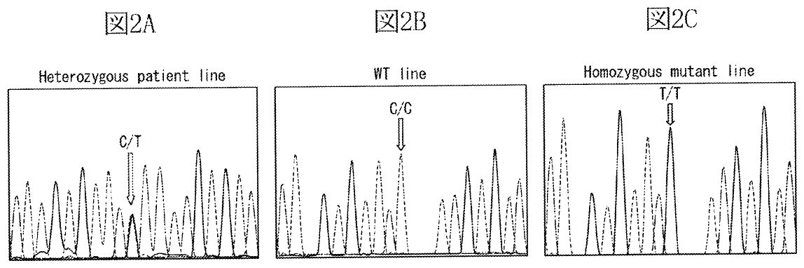

After genome editing, drug selection was performed, and colonies of surviving iPS cells were picked up to analyze the nucleotide sequence of the mutated site of the MAPT gene. FIGS. 2A to 2C show traces of the results of nucleotide sequence analysis. FIG. 2A shows the results obtained from a heterozygous mutant cell rat an iPS cell line, derived from a patient, FIG. 2B shows the results obtained from an isogenic wild-type cell of an iPS cell line, prepared by genome editing, and FIG. 2C shows the results obtained from an isogenic homozygous mutant cell of an iPS cell line, prepared by genome editing.

As a result of the analysis, it was confirmed that the isogenic wild-type S cell line and isogenic homozygous mutant iPS cell line were obtained by genome editing of the mutation in the mutated MAPT gene. Thereafter, each type of the IPS cells after the genome editing was transfected with PiggyBac transposase to remove the selection cassette. Subsequently, DNA sequencing confirmed that integration into the genomic DNA and other unintended mutations were not introduced.

### Experimental Example 3

(Preparation of Tau-Related Disease Model)

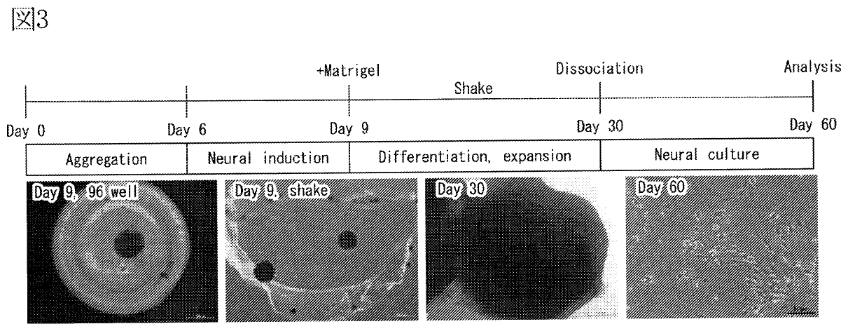

Each iPS cell line prepared in Experimental Example 2 was differentiated into neurons. FIG. 3 shows a schematic view of a schedule of differentiation to neurons and photographs of the cells.

First, a nerve organoid was prepared by three-dimensional culture for 30 days. Specifically, first, iPS cells were dissociated into single cells on day 0 and seeded in a low-adsorption V-type 96-well plate (Sumitomo Bakelite) at 3×10⁻⁵ cells/well to form embryoid bodies. The medium cased was StemFit AK02N medium (Ajinomoto) supplemented with 30 μM Y-27632, 5 μM SB-431542 and 2.5 μM IWP-2. Subsequently, the embryoid bodies were cultured for 6 days and induced into the forebrain of neural tissue.

Subsequently, on day 6 of culture, the medium was replaced with a nerve induction medium. The composition of the nerve induction medium was DMEM/Ham's F12 medium (Thermo Fisher Scientific Inc.) containing 1 (v/v) % N2 supplement (Thermo Fisher Scientific Inc.), 1 (v/v) % Glutamax (Thermo Fisher Scientific Inc.), 1 (v/v) % non-essential amino acid-MEM, 2.5 μM IWP-2, 5 μg/mL sodium heparan sulfate (Sigma-Aldrich Inc.) and 1 (v/v) % penicillin-streptomycin (Thermo Fisher Scientific Inc.).

Subsequently, on day 9 of culture, the culture was transferred to a low-adsorption 6-well plate (Corning Inc.) and cultured in a differentiation medium. The composition of the differentiation medium was a 1:1 mixed medium of DMEM/Ham's F12 medium (Thermo Fisher Scientific Inc.) and Neurobasal medium (Thermo Fisher Scientific Inc.) containing 1 (v/v) % N2 supplement (Thermo Fisher Scientific Inc.), 2 (v/v) % vitamin A-free B27 supplement (Thermo Fisher Scientific Inc.), 100 μM 2-mercaptoethanol, 2.5 μg/mL insulin (Fujifilm Wako Pure Chemical Industries, Ltd.), (v/v) % Glutamax (Thermo Fisher Scientific Inc.), 0.5(v/v) % non-essential amino acid-MEM and 1 (v/v) % Matrigel (registered trademark, Corning Inc.). Plates containing embryoid bodies were maintained on a shaker to facilitate absorption of nutrients and oxygen.

Subsequently, on day 15 of culture, the medium was replaced with a differentiation medium containing vitamin A-containing B27 supplement (Thermo Fisher Scientific Inc.). Thereafter, the medium was replaced every 5 to 7 days.

Subsequently, on day 30 of culture, the nerve organoid was dissociated into single cells using a neuron dispersion liquid (KAC Co., Ltd.). Subsequently, 5×10⁴, 1×10⁵ and 5×10⁵ cells were seeded in a 96-well plate, a 48-well plate and a 12-well plate coated with 60 poly-L-ornithine and 10 μg/mL laminin, respectively, and cultured for an additional 30 days, The medium used was Neurobasal medium (Thermo Fisher Scientific Inc.) containing 1 (v/v) % vitamin A-containing B27 supplement (Thermo Fisher Scientific Inc.), 0.25 (v/v) % Glutamax (Thermo Fisher Scientific Inc.) and 1 (v/v) % penicillin-streptomycin (Thermo Fisher Scientific Inc.). As a result, a two-dimensional culture of cells was obtained.

Subsequently, the resulting cells were then evaluated by immunofluorescent staining. Specifically, MAP2, which is a neuronal marker, βIII-tubulin, which is a neuronal marker, TBR1, which is a forebrain marker (cortical neuronal marker), FOXG1, which is a forebrain marker (cortical neuronal marker), NEUN, which is a mature neuron marker, and tau protein were stained.

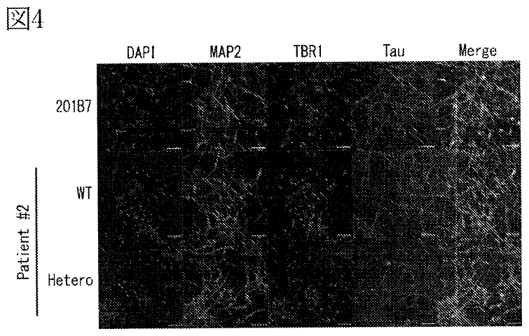

FIG. 4 shows fluorescence micrographs of the representative results of immunostaining of cells. The scale bar indicates 50 μm. In FIG. 4, “201B7” indicates the results of differentiating iPS cells derived from a healthy person, “Patient #2” indicates the results of differentiating IPS cells derived from patient #2, “WT” indicates the results for gene-edited isogonic wild-type, “Hetero” indicates the results for heterozygote of R406W mutant, and “Homo” indicates the results for gene-edited isogenic homozygote of R406W mutant.

As a result, it became apparent that the resulting cells were MAP2-positive, TBR1-positive and tau-positive, and were a pure neuron population. As will be described later, it became apparent that cells differentiated by the above method can be used as a tau-related disease model.

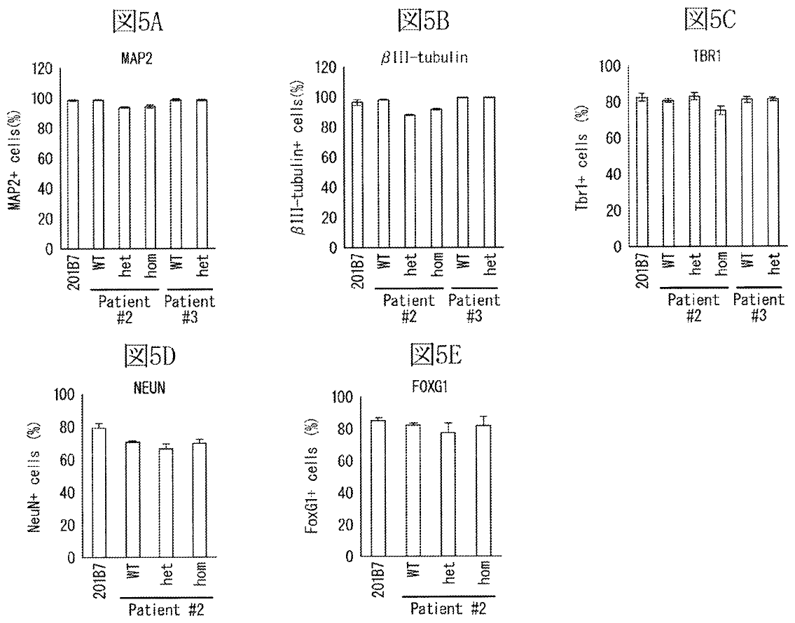

FIGS. 5A to 5E show graphs of the results of measuring the proportion of marker-positive cells. FIG. 5A shows the results for MAP2, FIG. 5B shows the results for βIII-tubulin, FIG. 5C shows the results for TBR1, and FIG. 5D shows the results for NEUN, and FIG. 5E shows the results for FOXG1. As a result, it became apparent that more than 85% of the neurons differentiated from each type of the iPS cells were MAP2-positive and βIII-tubulin-positive and were neurons. Further, it also became apparent that each type of these cells was TBR1-positive, FOXG1-positive and NEUN-positive. Therefore, each type of these cells was identified as cortical neurons.

### Experimental Example 4

(R406W Mutant Tau Protein Showed Reduced Degree of Phosphorylation by Various Kinases)

Subsequently, the phosphorylation of tau protein in cortical neurons was examined. In neurodegenerative diseases such as Alzheimer's disease and frontotemporal dementia, tau protein is known to be highly phosphorylated at multiple sites. On the other hand, interestingly, it has been reported that the R406W mutant tau protein has a reduced degree of phosphorylation at specific positions near the mutated site.

In this Experimental Example, phosphorylations of S404 (serine residue at position 404) and S409 (serine residue at position 409) in a tau-related disease model prepared in the same manner as in Experimental Example 3 were analyzed by Western blotting.

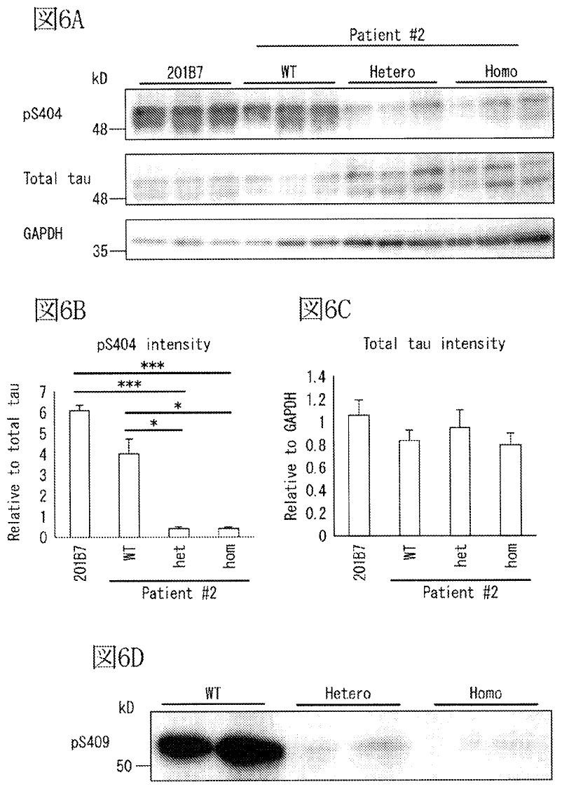

FIG. 6A shows a photograph of the results of examining phosphorylation of S404. Further, FIGS. 6B and 6C show graphs quantifying the results of FIG. 6A. In FIG. 6B, “*” indicates that there is a significant difference at p<0.05 by a Student's t-test, and “***” indicates that there is a significant difference at p<0.001. Further, FIG. 6C shows a photograph of the results of examining phosphorylation of S409. Further, FIG. 6D shows a photograph of the results of examining phosphorylation of S409.

In FIGS. 6A to 6D, “pS404” indicates the results of staining with an antibody against tau protein in which S404 is phosphorylated, and “pS409” indicates the results of staining with an antibody against tau protein in which S409 is phosphorylated, “201B7” indicates the results of differentiating iPS cells derived from a healthy person, “Patient #2” indicates the results of differentiating iPS cells derived from patient #2, “WT” indicates the results for gene-edited isogenic wild-type, “Hetero” indicates the results for heterozygote of R406W mutant, “Homo” indicates the results for gene-edited isogenic homozygote R406W mutant, “Total tau” indicates the results of staining all tau proteins, and “GAPDH” indicates the results of detecting glyceraldehyde-3-phosphate dehydrogenase protein.

As a result, it became apparent that the degree of phosphorylation of S404 and S409 was significantly reduced in both the heterozygote and the homozygote of R406W mutant, relative to the wild-type.

### Experimental Example 5

(R406W Mutant Tau was Cleaved at the C-Terminal Side)

As a result of detecting R406W mutant tau protein by Western blotting in a tau-related disease model prepared in the same manner as in Experimental Example 3, it became apparent that the R406W mutant tau protein was cleaved to produce fragments ranging from 35 kD to 45 kD in size. This was particularly noticeable in the case where Western blotting was performed using a Tau12 antibody recognizing the N-terminus of tau protein. Therefore, it was considered the C-terminal side of the tau protein was cleaved and fragmented.

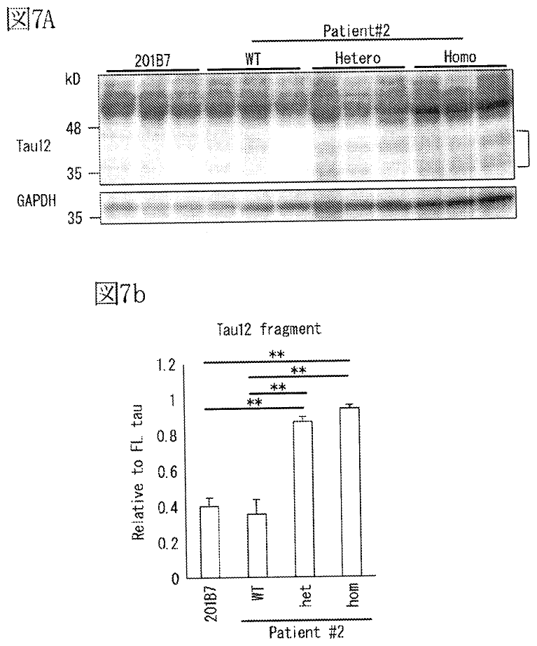

FIG. 7A shows photographs of the results of Western blotting using a Tau12antibody. Further, FIG. 7B shows a graph quantifying the amounts of tau protein fragments ranging from 35 kD to 45 kD in size based on the results of FIG. 7A. In FIGS. 7A and 7B, “201B7” indicates the results of differentiating iPS cells derived from a healthy person, “Patient #2” indicates the results of differentiating iPS cells derived from patient #2, “WT” vindicates the results for gene-edited isogenic wild-type, “Hetero” indicates the results for heterozygote of R406W mutant, “Homo” indicates the results for gene-edited isogenic homozygote R406W mutant, and “GAPDH” indicates the results of detecting glyceraldehyde-3-phosphate dehydrogenase protein. Further, in FIG. 7B, “**” indicates that there is a significant difference at p<0.01 by a Student's t-test.

As a result, it became apparent that fragmented tau protein was significantly increased in both the heterozygote and the homozygote of R406W mutants, relative to the wild-type.

### Experimental Example 6

(Examination of Phenotype of Mutant Neurons)

Using a tau-related disease model prepared in the same manner as in Experimental Example 3, cell phenotypes induced by R406W mutant tau protein were examined

First, neurons of the tau-related diseases model were immunostained and analyzed by In Cell Analyzer 6000 (GE Healthcare) to examine co-localization of MAP2 and tau protein.

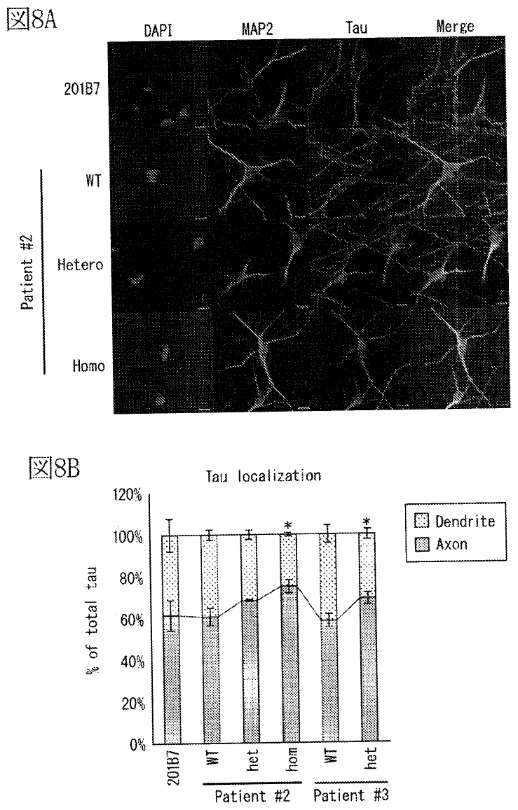

FIG. 8A shows fluorescence micrographs of the representative results of immunostaining. The scale bar indicates 10 μm. In FIG. 8A, “201B7” indicates the results of differentiating iPS cells derived from a healthy person, “Patient #2” indicates the results of differentiating iPS cells derived from patient #2, “WT” indicates the results for gene-edited isogenic wild-type, “Hetero” indicates the results for heterozygote of R406W mutant, “Homo” indicates the results for gene-edited isogenic homozygote of R406W mutant, “DAPI” indicates the results of staining the nuclei with 4′,6-diamidino-2-phenylindole, and “Merge” indicates the results from overlapping the images.

As a result, it became apparent that the R406W mutants had an increased proportion of co-localization of MAP2 and tau proteins.

Further, FIG. 8B shows a graph quantifying proportions of tau protein localized in dendrites, and is a graph quantifying proportions of tau protein localized in axons based on the results of the immunostaining. Thu protein localized in the MAP2-positive region was measured as tan protein localized in dendrites. Further, tau protein localized in βIII-tubulin-positive and MAP2-negative regions measured as tau protein localized in axons.

In FIG. 8B, “201B7” indicates the results of differentiating iPS cells derived from a healthy person, “Patient #2” indicates the results of differentiating iPS cells derived from patient #2, “Patient #3” indicates the results of differentiating iPS cells derived from patient #3, “WT” indicates the results for gene-edited isogenic wild-type, “het” indicates the results for heterozygote of R406W mutant, “hom” indicates the results for gene-edited isogenic homozygote of R406W mutant, “Dendrite” indicates the proportion of tau protein localized in dendrites, an “Axon” indicates the proportion of tau protein localized in axons. Further, “*” indicates that there is a significant difference at p<0.05 by a Student's t-test.

As a result, it became apparent that the proportion of the mutant tau protein localized in MAP2-positive dendrites increased. From these results, it became apparent that the R406W mutant tau protein tends to be mislocalized in dendrites rather than in axons where the protein is originally localized.

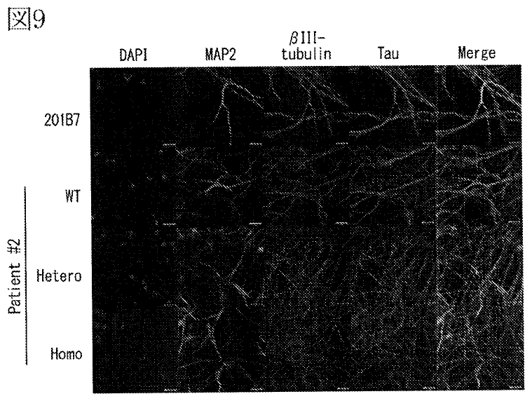

Further, the morphology of neurons was examined by immunostaining. FIG. 9 shows fluorescence micrographs of the representative results of immunostaining. The scale bar indicates 20 μm. In FIG. 9, “201B7” indicates the results of differentiating iPS cells derived from a healthy person, “Patient #2” indicates the results of differentiating iPS cells derived from patient #2, “WT” indicates the results for gene-edited isogenic wild-type, “Hetero” indicates the results for heterozygote of R406W mutant, “Homo” indicates the results for gene-edited isogenic homozygote of R406W mutant, “DAPI” indicates the results of staining the nuclei with 4′,6-diamidino-2-phenylindole, and “Merge” indicates the results from overlapping the images.

As shown in FIG. 9, βIII-tubulin taming revealed microtubule degeneration in R406W mutant neurons. Then, it became apparent that the axons of the mutant neurons were composed of a large number of small puncta.

### Experimental Example 7

(Examination of Microtubule Destabilization)

A tau-related disease model prepared in the same manner as in Experimental Example 3 was treated with a microtubule-stabilizing agent, epothilone D (EpoD) and βIII-tubulin was stained by immunostaining. Specifically, tau-related disease models were incubated for 24 hours in the presence and in the absence of Epothilone D (Abeam) at a final concentration of 20 nM, followed by immunostaining.

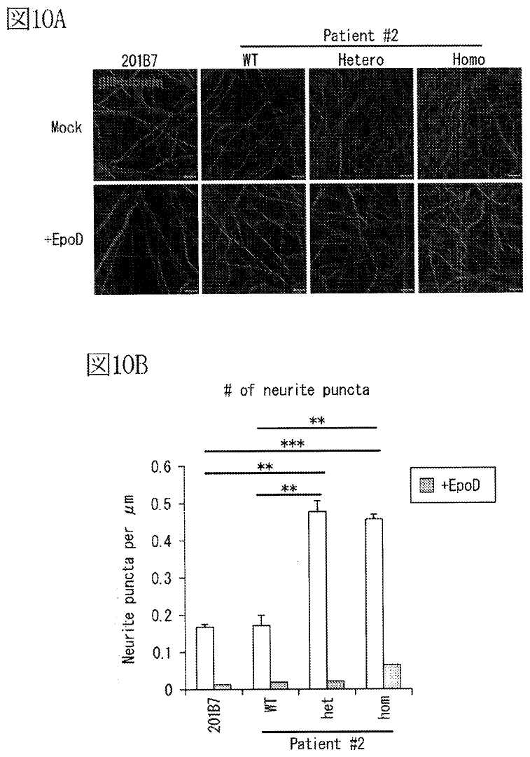

FIG. 10A shows fluorescence micrographs of the representative results of immunostaining. The scale bar indicates 10 μm. In FIG. 10A, “201B7” indicates the results of differentiating iPS cells derived from a healthy person, “Patient #2” indicates the results of differentiating iPS cells derived from patient #2, “WT” indicates the results for gene-edited isogenic wild-type, “Hetero” indicates the results for heterozygote of R406W0 mutant, and “Homo” indicates the results for gene-edited isogenic homozygote of R406W mutant. Further, “+EpoD” indicates the result of the epothilone D treatment.

Further, FIG. 10B shows a graph of the results of measuring the number of neurite puncta based on the results of βIII-tubulin immunostaining. In FIG. 10B, “201B7” indicates the results of differentiating iPS cells derived from a healthy person, “Patient #2” indicates the results of differentiating iPS cells derived from patient #2, “WT” indicates the results for gene-edited isogenic wild-type, “het” indicates the results for heterozygote of R406W mutant, “hom” indicates the results for gene-edited isogenic homozygote of R406W mutant, and “+EpoD” indicates the results of epothilone D treatment. Further, “**” indicates that there is a significant difference at p<0.01 by a Student's t-test, and “***” indicates that there is a significant difference at p<0.001. The vertical axis of the graph represents the number of neurite puncta per μm.

As a result, it became apparent that the epothilone D treatment rescued the degeneration of microtubules.

From the above results, it became apparent that the R406W mutant tau protein changes the dynamics or stability of microtubules and thereby induces axonal degeneration.

### Experimental Example 8

(Examination of Mitochondrial Transport in Mutant Neurons)

Mitochondrial transport in mutant neurons was examined. First, the expression vector of Mito-eYFP and the expression vector of tdTomato were introduced into a tau-related disease model prepared in the same manner as in Experimental Example 3. Subsequently, the number of mitochondria on the axon was measured by fluorescence microscopy. Mito-eYFP is a fluorescent protein that is specifically labels mitochondria.

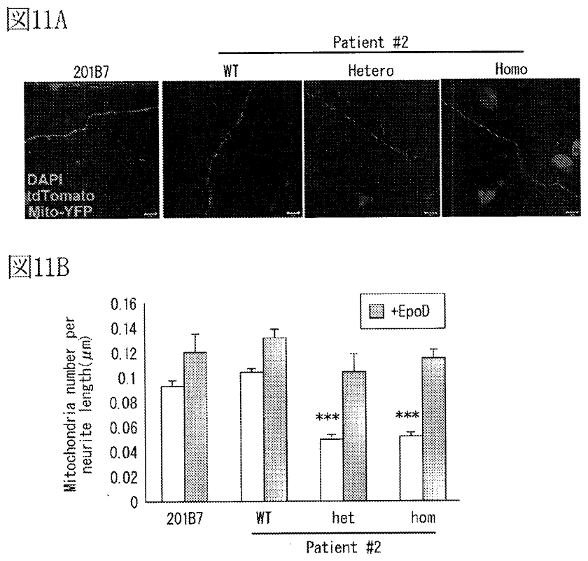

FIG. 11A shows representative fluorescence micrographs. The scale bar indicates 10 μm. Further, FIG. 11B shows a graph of the results of measuring the number of mitochondria present on axons based on the fluorescence micrographs.

In FIGS. 11A and 11B, “201B7” indicates the results of differentiating iPS cells derived from a healthy person, “Patient #2” indicates the results of differentiating iPS cells derived from patient #2, “WT” indicates the results for gene-edited isogenic wild-type, “Hetero” indicates the results for heterozygote of R406W mutant, “Homo” indicates the results for gene-edited isogenic homozygote of R406W mutant, and “+EpoD” indicates the results of epothilone D treatment. Further, “***” indicates that there is a significant difference at p<0.001 by a Student's t-test. The vertical axis of the graph represents the number of mitochondria per μm of neurite.

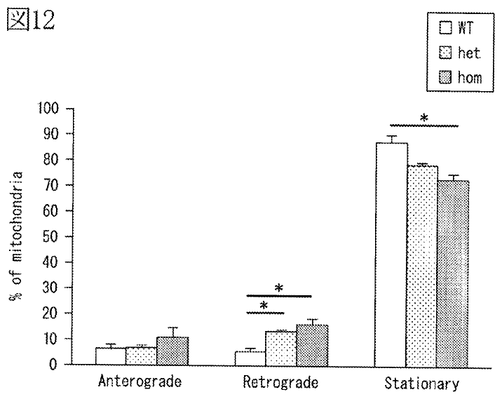

Further, FIG. 12 shows a graph of the results of measurement of the proportion of anterograde, retrograde, and stationary mitochondria by live image analysis using a confocal laser microscope (model number “FV3000”, Olympus). In FIG. 12, “WT” indicates the results for gene-edited isogenic wild-type, “het” indicates the results for heterozygote of R406W mutant, “hom” indicates the results for gene-edited isogenic homozygote of R406W mutant, “Anterograde” indicates the results for anterograde mitochondria, “Retrograde” indicates the results for retrograde mitochondria, and “Stationary” indicates stationary mitochondria. Further, “*” indicates that there is a significant difference at p<0.05 by a Student's t-test. The vertical axis of the graph represents the proportion (%) of mitochondria.

As a result, it became apparent that the proportion of stationary mitochondria was lower and the proportion of retrograde mitochondria was higher in the mutant neurons than in the wild-type neurons. These results indicate that microtubule destabilization induced by mutant tau protein is responsible for the dysfunction of axonal transport mechanisms.

### INDUSTRIAL APPLICABILITY

According to the present invention, a technique for producing a tau-related disease model can be provided.

1. A method for producing a tau-related disease model, the method comprising:
three-dimensionally culturing pluripotent stem cells having a mutation in Microtubule Associated Protein Tau (MAPT) gene to form a nerve organoid; and
dissociating the nerve organoid into single cells and performing two-dimensional adherent culture to obtain neurons,
wherein the neurons are a tau-related disease model.

2. The method for producing according to claim 1,
wherein the mutation is one or a plurality of mutations present in exons 9 to 13 or intron 10.

3. The method for producing according to claim 2,
wherein the mutation present in exons 9 to 13 is a mutation that encodes a mutant tau protein selected from the group consisting of K257T, I260V, G272V, N279K, K280Δ, L284L, S285R, N296H, P301L, P301S, S305N, S303S, S305S, V337M, E342V, G389R and R406W, and
the mutation present in intron 10 is a mutation in any one or a plurality of bases at nucleotide positions 1 to 20 from the 5′-end of intron 10.

4. A tau-related disease model comprising neurons having a mutation in the MAPT gene,
wherein the neurons are significantly different in the degree of phosphorylation of tau protein, degree of fragmentation of tau protein, degree of axonal degeneration or axonal transport function, relative to wild-type neurons.

5. The tau-related disease model according to claim 4, which is produced by the method for producing a tau-related disease model, the method comprising:
three-dimensionally culturing pluripotent stem cells having a mutation in MAPT gene to form a nerve organoid; and
dissociating the nerve organoid into single cells and performing two-dimensional adherent culture to obtain neurons,
wherein the neurons are a tau-related disease model.

6. A method for screening a preventive or therapeutic agent for a tau-related disease, the method comprising:
culturing the tau-related disease model according to claim 4 in the presence of a test substance; and
measuring the degree of phosphorylation of tau protein, degree of fragmentation of tau protein, degree of axonal degeneration or axonal transport function of the tau-related disease model,
wherein a significant difference in the measured degree of phosphorylation of tau protein, degree of fragmentation of tau protein, degree of axonal degeneration or axonal transport function, relative to that in the absence of the test substance, is an indication that the test substance is a preventive or therapeutic agent for a tau-related disease.

7. A method for screening a preventive or therapeutic agent for a tau-related disease, the method comprising:
culturing the tau-related disease model according to claim 5 in the presence of a test substance; and
measuring the degree of phosphorylation of tau protein, degree of fragmentation of tau protein, degree of axonal degeneration or axonal transport function of the tau-related disease model,
wherein a significant difference in the measured degree of phosphorylation of tau protein, degree of fragmentation of tau protein, degree of axonal degeneration or axonal transport function, relative to that in the absence of the test substance, is an indication that the test substance is a preventive or therapeutic agent for a tau-related disease.
